(19) United States
(12) Patent Application Publication
(10) Pub. No.: US 2019/0030530 A1
(43) Pub. Date: Jan. 31, 2019

(54) MICRO- AND NANOCONTACT PRINTING WITH AMINOSILANES: PATTERNING SURFACES OF MICROFLUIDIC DEVICES FOR MULTI- PLEXED BIOASSAYS

(71) Applicant: OKINAWA INSTITUTE OF SCIENCE AND TECHNOLOGY SCHOOL CORPORATION, Okinawa (JP)

(72) Inventors: Amy Shen FRIED, Okinawa (JP); Sebastien Georg Gabriel RICOULT, Okinawa (JP)

(21) Appl. No.: 16/073,791

(22) PCT Filed: Feb. 1, 2017
(86) PCT No.: PCT/JP2017/003621

§ 371 Date: Jul. 29, 2018

Related U.S. Application Data
(60) Provisional application No. 62/290,067, filed on Feb. 2, 2016.

(51) Int. Cl. B01L 3/00; G01N 33/543; G01N 33/552

(52) U.S. Cl. CPC B01L 3/502707; G01N 33/54353; G01N 33/54393; B01L 2300/123; B01L 2300/0636; B01L 2300/0896; G01N 33/552

(57) ABSTRACT

It is an object of the present invention to achieve rapid surface patterning of biomolecules within microfluidic devices with high reproducibility. In this work, we present a new means of creating micro- and nano-patterns of aminosilanes within microfluidic devices via an aqueous based microcontact printing technique. To minimize the diffusion of molecules into the PDMS stamp, we use water as the inking solvent and enforce short incubation and contact times during the printing process to preserve the predefined resolution of patterned features. These patterns then serve as the building block to couple multiple biomolecules in solution onto a single surface for subsequent bioassays.

### TECHNICAL FIELD

The invention is related to the area of micro- and nanocontact printing. In particular, it is related to micro- and nanocontact printing with aminosilanes: patterning surfaces of microfluidic devices for multi-plexed bioassays.

### BACKGROUND ART

Since the early 1980's, microfluidic systems have advanced significantly to satisfy the growing demand for the miniaturization of bioassay devices with applications ranging from disease diagnostics (W. Su, X. Gao, L. Jiang, and, J. Qin, Journal of Chromatography A, 2015, 1377, 13-26; D. G. Rackus, M. H. Shamsi and A. R. Wheeler, Chemical Society Reviews, 2015, 44, 5320-5340.; M. Karle, S. K. Vashist, R. Zengerle and F. von Stetten, Analytica Chimica Acta, 2016, 929, 1-22.; V. C. Rucker, K. L. Havenstrite, B. A. Simmons, S. M. Sickafoose, A. E. Herr and R. Shediac, Langmuir, 2005, 21, 7621- 7625.) to cell behavior studies (G. Du, Q. Fang and J. M. den Toonder, Analytica Chimica Acta, 2016, 903, 36-50.; A. Karimi, D. Karig, A. Kumar and A. Ardekani, Lab on a Chip, 2015, 15, 23-42.; N. D. Gallant, J. L. Charest, W. P. King and A. J. Garcia, Journal of nanoscience and nanotechnology, 2007, 7, 803-807.; S. Takayama, J. C. McDonald, E. Ostuni, M. N. Liang, P. J. Kenis, R. F. Ismagilov and G. M. Whitesides, Proceedings of the National Academy of Sciences, 1999, 96, 5545-5548.), with the goal to provide cheaper, simpler and more reliable means for simultaneous analysis of multiple biosensing reactions (P. Angenendt, J. Glokler, Z. Konthur, H. Lehrach and D. J. Cahill, Analytical chemistry, 2003, 75, 4368-4372.; S. Choi, M. Goryll, L. Y. M. Sin, P. K. Wong and J. Chae, Microfluidics and Nanofluidics, 2011, 10, 231-247.; B. S. Munge, T. Stracensky, K. Gamez, D. DiBiase and J. F. Rusling, Electroanalysis, 2016.; C. K. Tang, A. Vaze, M. Shen and J. F. Rusling, ACS sensors, 2016, 1, 1036-1043.). Out of the available platforms, surface-based microfluidic bioassay devices are rising to the forefront, owing to the enhanced sensitivity and ease of detection provided by these systems, largely attributed by the precise control of reaction sites by surface patterning (M. Zimmermann, E. Delamarche, M. Wolf and P. Hunziker, Biomedical microdevices, 2005, 7, 99-110.). Notably, the performance of these technologies is highly influenced by the quality of biomolecule surface patterning, ie., the surface density, orientation, and biofunctionality of patterned biomolecules. Additionally, the ease of operation, handling, and integration of the surface patterns into the devices is another important factor that influences the overall impact of these systems.

Existing techniques to pattern biomolecules on surfaces at the micro- and nanoscales, include physical patterning approaches such as photolithography (E. E. Hui and S. N. Bhatia, Langmuir, 2007, 23, 4103-4107.), adsorption of biomolecules confined to microfluidic networks (J. L. Garcia-Cordero and S. J. Maerkl, Chemical Communications, 2013, 49, 1264-1266.), and colloidal lithography (M. A. Ray, N. Shewmon, S. Bhawalkar, L. Jia, Y. Yang, and E. S. Daniels, Langmuir, 2009, 25, 7265-7270.). These techniques are either plagued by high costs, low throughput, or limited control over the geometry and functional properties of the achieved patterns. Particularly, nanopatterning of biomolecules has been laborious and integration of these patterned substrates into microfluidic devices has been a challenge. A recent report proposed a self-assembly-based colloidal lithography technique to generate nanopatterns which were then sealed into PDMS microchannels to immobilize proteins onto the nanopatterns via non-covalent coupling (A. S. Andersen, W. Zheng, D. S. Sutherland and X. Jiang, Lab on a Chip, 2015, 15, 4524-4532.). Although the proposed method enables the successful generation of multiple protein nanopatterns within a microfluidic channel, the major drawbacks are the requirement of complex fabrication techniques to create the nanopatterned substrates, the repeated fabrication of new surfaces prior to each use, and the non-covalent coupling of proteins onto the nanopatterns inducing potential desorption when subjected to flow.

One of the simpler and preferred methods of patterning micro- and nanoscale features is microcontact printing (μCP), where chemical or biological molecules are transferred in designated patterns from an elastomeric poly(dimethylsiloxane) (PDMS) stamp onto a substrate with higher surface energy (M. Mrksich and G. M. Whitesides, Trends in biotechnology, 1995, 13, 228-235.; L. Filipponi, P. Livingston, O. Kaspar, V. Tokarova and D. V. Nicolau, Biomedical microdevices, 2016, 18, 1-7.; R. Castagna, A. Bertucci, E. A. Prasetyanto, M. Monticelli, D. V. Conca, M. Massetti, P. P. Sharma, F. Damin, M. Chiari, L. De Cola et al., Langmuir, 2016, 32, 3308-3313.). Although these microcontact printed biomolecules have been successfully incorporated into microfluidic devices (R. S. Kane, S. Takayama, E. Ostuni, D. E. Ingber and G. M. Whitesides, Biomaterials, 1999, 20, 2363-2376.; E. B. Chakra, B. Hannes, J. Vieillard, C. D. Mansfield, R. Mazurczyk, A. Bouchard, J. Potempa, S. Krawczyk and M. Cabrera, Sensors and Actuators B: Chemical, 2009, 140, 278-286.), several challenges remain. First, as patterned biomolecules are physically adsorbed onto the surfaces driven by hydrogen-bonding and van der Waals forces (W. Norde, Colloids and Surfaces B: Biointerfaces, 2008, 61, 1-9.), they are unable to withstand high shear stresses introduced by the flow present in microfluidic channels. As a result, it gives rise to gradual desorption and degradation of patterned biomolecules that lead to reduced device performance and poor shelf life. Secondly, since partial dehydration of biomolecules is a prerequisite to the μCP technique, the probability of protein denaturation and impaired biological activity is high.

Additionally, the lack of control over the orientation of the printed proteins has been lamented and could be responsible for the suboptimal interactions in bioassays due to the inaccessibility of the binding sites. Lastly, patterning a substrate with multiple biomolecules proves to be difficult and time-consuming, as each individual stamp can only be utilized to pattern a single ink at a time.

To address these challenges, pre-patterned substrates have been used to covalently link the biomolecules from solution to pattern sensitive biomolecules. For example, Teerapanich, et al. recently achieved real-time monitoring of protein-binding kinetics by creating patterned gold films to stably and covalently immobilize antibodies within nanofluidic channels (P. Teerapanich, M. Pugniere, C. Henriquet, Y.-L. Lin, C.-F. Chou and T. Leichle, Biosensors and Bioelectronics, 2016). Others have reported a simpler and more stable surface patterning technique that employs covalent coupling of proteins and nucleotides to silane treated substrates (Y. Wu, T. Buranda, R. L. Metzenberg, L. A. Sklar and G. P. Lopez, Bioconjugate Chemistry, 2006, 17, 359-365.; H. H. Weetall, Applied Biochemistry and Biotechnology, 1993, 41, 157-188). Recently, Lin et al. demonstrated a novel method of covalently patterning multiple proteins to a (3-glycidyloxypropyl) trimethoxysilane modified substrate enclosed within nanochannels by using a robotic microarray spotter (Y.-L. Lin, Y.-J. Huang, P. Teerapanich, T. Leichle and C.-F. Chou, Biomicrofluidics, 2016, 10, 034114.). However, alignment of the channels with the patterns is difficult to achieve since the proteins are deposited onto the substrates prior to the bonding of the device. Additionally, as the proteins are dried briefly before alignment, viability for long-term studies is a concern due to their potential degradation.

Alternatively, (3-aminopropyl)triethoxysilane (APTES), an amine-NH2 terminated silane can be used to form covalent siloxane bonds with silica substrates under pertinent conditions (N. Aissaoui, L. Bergaoui, J. Landoulsi, J.-F. Lambert and S. Boujday, Langmuir, 2011, 28, 656-665.). Anhydrous organic solvents like toluene have been widely used to achieve homogeneity of the formed monolayers and to ensure covalent binding of APTES with the glass substrates (R. M. Pasternack, S. Rivillon Amy and Y. J. Chabal, Langmuir, 2008, 24, 12963-12971.). These terminal amine groups then serve to covalently couple biomolecules with the help of appropriate linkers (S. K. Vashist, E. Lam, S. Hrapovic, K. B. Male and J. H. Luong, Chemical reviews, 2014, 114, 11083-11130.). Several studies have demonstrated the potential of μCP to create patterns of APTES monolayers within microfluidic channels that are then covalently coupled with biomolecules from solution (T. F. Didar, A. M. Foudeh and M. Tabrizian, Analytical chemistry, 2011, 84, 1012-1018.; G. Arslan, M. Ozmen, I. Hatay, I. H. Gubbuk and M. Ersoz, Turkish Journal of Chemistry, 2008, 32, 313-321.). Although this method provides simplicity and potential for achieving multiplexing in microfluidic devices, the resolution of obtained features is not only limited by the microfluidic channel dimensions, but also by the printing process since existing μCP methods rely on the use of organic solvents that can potentially swell the PDMS substrate and increase the dimensions of the patterned features (J. N. Lee, C. Park and G. M. Whitesides, Analytical Chemistry, 2003, 75, 6544-6554). Although the degree of PDMS swelling does not significantly affect micron-size features in the stamps, it proves to be a limiting factor while attempting to achieve nanoscale APTES patterns. Notably, similar to thiols, silanes being small molecules, can diffuse into the PDMS stamp upon long incubation times (T. E. Balmer, H. Schmid, R. Stutz, E. Delamarche, B. Michel, N. D. Spencer and H. Wolf, Langmuir, 2005, 21, 622-632.). As a result, during the printing step (on the order of minutes), silane molecules tend to diffuse out of the stamp along with the solvent molecules, reducing the resolution of the patterned features (Y. Xia and G. M. Whitesides, Annual review of materials science, 52. 1998, 28, 153-184.).

### CITATION LIST

### Non Patent Literature

### NPL 1: W. Su, X. Gao, L. Jiang and J. Qin, Journal of Chromatography A, 2015, 1377, 13-26.

### NPL 2: D. G. Rackus, M. H. Shamsi and A. R. Wheeler, Chemical Society Reviews, 2015, 44, 5320-5340.

NPL 3: M. Karle, S. K. Vashist, R. Zengerle and F. von Stetten, Analytica chimica acta, 2016, 929, 1-22.

### NPL 4: V. C. Rucker, K. L. Havenstrite, B. A. Simmons, S. M. Sickafoose, A. E. Herr and R. Shediac, Langmuir, 2005, 21, 7621-7625.

NPL 5: G. Du, Q. Fang and J. M. den Toonder, Analytica chimica acta, 2016, 903, 36-50.

### NPL 6: A. Karimi, D. Karig, A. Kumar and A. Ardekani, Lab on a Chip, 2015, 15, 23-42.

NPL 7: N. D. Gallant, J. L. Charest, W. P. King and A. J. Garcia, Journal of nanoscience and nanotechnology, 2007, 7, 803-807.

### NPL 8: S. Takayama, J. C. McDonald, E. Ostuni, M. N. Liang, P. J. Kenis, R. F. Ismagilov and G. M. Whitesides, Proceedings of the National Academy of Sciences, 1999, 96, 5545-5548.

NPL 9: P. Angenendt, J. Glokler, Z. Konthur, H. Lehrach and D. J. Cahill, Analytical chemistry, 2003, 75, 4368-4372.

### NPL 10: S. Choi, M. Goryll, L. Y. M. Sin, P. K. Wong and J. Chae, Microfluidics and Nanofluidics, 2011, 10, 231-247.

### NPL 11: B. S. Munge, T. Stracensky, K. Gamez, D. DiBiase and J. F. Rusling, Electroanalysis, 2016.

NPL 12: C. K. Tang, A. Vaze, M. Shen and J. F. Rusling, ACS sensors, 2016, 1, 1036-1043.
NPL 13: M. Zimmermann, E. Delamarche, M. Wolf and P. Hunziker, Biomedical microdevices, 2005, 7, 99-110.

### NPL 14: E. E. Hui and S. N. Bhatia, Langmuir, 2007, 23, 4103-4107.

### NPL 15: J. L. Garcia-Cordero and S. J. Maerkl, Chemical Communications, 2013, 49, 1264-1266.

### NPL 16: M. A. Ray, N. Shewmon, S. Bhawalkar, L. Jia, Y. Yang and E. S. Daniels, Langmuir, 2009, 25, 7265-7270.

### NPL 17: A. S. Andersen, W. Zheng, D. S. Sutherland and X. Jiang, Lab on a Chip, 2015, 15, 4524-4532.

NPL 18: M. Mrksich and G. M. Whitesides, Trends in biotechnology, 1995, 13, 228-235.
NPL 19: L. Filipponi, P. Livingston, O. Kaspar, V. Tokarova and D. V. Nicolau, Biomedical microdevices, 2016, 18, 1-7.

### NPL 20: R. Castagna, A. Bertucci, E. A. Prasetyanto, M. Monticelli, D. V. Conca, M. Massetti, P. P. Sharma, F. Damin, M. Chiari, L. De Cola et al., Langmuir, 2016, 32, 3308-3313.

### NPL 21: R. S. Kane, S. Takayama, E. Ostuni, D. E. Ingber and G. M. Whitesides, Biomaterials, 1999, 20, 2363-2376.

### NPL 22: E. B. Chakra, B. Hannes, J. Vieillard, C. D. Mansfield, R. Mazurczyk, A. Bouchard, J. Potempa, S. Krawczyk and M. Cabrera, Sensors and Actuators B: Chemical, 2009, 140, 278-286.

### NPL 23: W. Norde, Colloids and Surfaces B: Biointerfaces, 2008, 61, 1-9.

### NPL 24: P. Teerapanich, M. Pugniere, C. Henriquet, Y.-L. Lin, C.-F. Chou and T. Leichle, Biosensors and Bioelectronics, 2016.

### NPL 25: Y. Wu, T. Buranda, R. L. Metzenberg, L. A. Sklar and G. P. Lopez, Bioconjugate Chemistry, 2006, 17, 359-365.

### NPL 26: H. H. Weetall, Applied Biochemistry and Biotechnology, 1993, 41, 157-188.

### NPL 27: Y.-L. Lin, Y.-J. Huang, P. Teerapanich, T. Leichle and C.-F. Chou, Biomicrofluidics, 2016, 10, 034114.

### NPL 28: N. Aissaoui, L. Bergaoui, J. Landoulsi, J.-F. Lambert and S. Boujday, Langmuir, 2011, 28, 656-665.

### NPL 29: R. M. Pasternack, S. Rivillon Amy and Y. J. Chabal, Langmuir, 2008, 24, 12963-12971.

NPL 30: S. K. Vashist, E. Lam, S. Hrapovic, K. B. Male and J. H. Luong, Chemical reviews, 2014, 114, 11083-11130.
NPL 31: T. F. Didar, A. M. Foudeh and M. Tabrizian, Analytical chemistry, 2011, 84, 1012-1018.

### NPL 32: G. Arslan, M. Ozmen, I. Hatay, I. H. Gubbuk and M. Ersoz, Turkish Journal of Chemistry, 2008, 32, 313-321.

### NPL 33: J. N. Lee, C. Park and G. M. Whitesides, Analytical Chemistry, 2003, 75, 6544-6554.

### NPL 34: T. E. Balmer, H. Schmid, R. Stutz, E. Delamarche, B. Michel, N. D. Spencer and H. Wolf, Langmuir, 2005, 21, 622-632.

NPL 35: Y. Xia and G. M. Whitesides, Annual review of materials science, 1998, 28, 153-184.

### SUMMARY OF INVENTION

### Technical Problem

As the functionality of surface-based microfluidic bioassay devices is determined by the efficiency and accuracy of surface patterning of biomolecules, there is an increasing demand for new technologies to create surface patterns at micro- and nanoscales. It is an object of the present invention to achieve rapid surface patterning of biomolecules within microfluidic devices with high reproducibility.

### Solution to Problem

In this work, we present a new means of creating micro- and nano-patterns of aminosilanes within microfluidic devices via an aqueous based microcontact printing technique. To minimize the diffusion of molecules into the PDMS stamp, we use water as the inking solvent and enforce short incubation and contact times during the printing process to preserve the pre-defined resolution of patterned features 36. These patterns then serve as the building block to couple multiple biomolecules in solution onto a single surface for subsequent bioassays. To validate the functionality of the coupled biomolecules, we carry out an aptamer based immunoassay to detect Interleukin 6 (IL6) and an antibody based immunoassay for the detection of human C-reactive protein (hCRP). We probe the stability of APTES patterns and demonstrate the possibility of fabricating pre-stored and ready-to-use bioassay devices with a shelf life of at least 3 months. Finally, we verify the multiplexing capability on a single patterned surface by delivering different biomolecules to different regions of the patterned array with the help of microfluidic networks and liquid dispensing technologies.

The present inventions are as follows.
[1] An array of biomolecules comprising a substrate and a probe molecule, wherein the surface of the substrate has patterned nano features of silane.
[2] The array of biomolecules according to [1], wherein the unpatterned surface of the substrate is blocked with PEG-silane.
[3] The array of biomolecules according to [1], wherein the diameter of the nano features is patterned from 10 nm to 1000 nm.
[4] The array of biomolecules according to [1], wherein the probe molecule is selected from the group consisting of proteins, peptides, antibodies, nucleic acids, carbohydrates and lipids
[5] The array of biomolecules according to [1], wherein the probe molecule is conjugated onto nano features of silane on the substrate.
[6] A kit comprising a substrate and a probe molecule, wherein the surface of the substrate has patterned nano features of silane.
[7] The kit according to [6], wherein the unpatterned surface of the substrate is blocked with PEG-silane.
[8] The kit according to [6], wherein the diameter of the nano features is patterned from 10 nm to 1000 nm.
[9] The kit according to [6], wherein the probe molecule is selected from the group consisting of proteins, peptides, antibodies, nucleic acids, carbohydrates and lipids.
[10] A substrate for an array of biomolecules, wherein the surface of the substrate has patterned nano features of silane for conjugating a probe molecule.
[11] The substrate for an array of biomolecules according to [10], wherein the unpatterned surface of the substrate is blocked with PEG-silane.
[12] The substrate for an array of biomolecules according to [10], wherein the diameter of the nano features is patterned from 10 nm to 1000 nm.
[13] The substrate for an array of biomolecules according to [10], wherein the biomolecule is selected from the group consisting of proteins, peptides, antibodies, nucleic acids, carbohydrates and lipids.
[14] A method of micro or nano patterning of biomolecules in a multiplex format comprising the steps of
   covering a silicone wafer (Si Wafer) which has nanoholes with PDMS;
   separating the PDMS from the Si Wafer;
   covering the PDMS with a photo-sensitive polymer;
   exposing the photo-sensitive polymer to light;
   separating the photo-sensitive polymer replica from the PDMS;
   contacting the photo-sensitive polymer replica which was plasma activated with a planar PDMS stamp which was incubated with silane;
   separating the flat PDMS to lift-off silane in the contact areas;
   printing the flat PDMS with nanoholes of silane onto a glass slide;
   separating the flat PDMS from the glass slide;
   incubating the patterned silane with an aqueous PEG silane to block the unpatterned surface; and then
   incubating the patterned/blocked APTES silane with desired biomolecule.
[15] The method of [14] wherein the biomolecule is selected from the group consisting of proteins, peptides, antibodies, nucleic acids, carbohydrates and lipids.
[16] The method of [14] wherein the diameter of nanoholes is patterned from 10 nm to 1000 nm.

### Advantageous Effects of Invention

The present invention of a simple aqueous based microcontact printing (μCP) method can create stable micro- and nanopatterns of (3-aminopropyl)triethoxysilane (APTES) on glass substrates of microfluidic devices with feature sizes ranging from a few hundred microns to 200 nm. By combining our surface patterning technique with sensing technologies, highly sensitive bioassay systems at nanoscale can be developed in the near future.

### BRIEF DESCRIPTION OF DRAWINGS

FIG. 1 Fabrication of patterned microfluidic devices. (a) Microcontact printing for APTESaq micropatterns: (i) a PDMS stamp inked with APTESaq is contacted with a plasma activated glass surface through microcontact printing to (ii) transfer APTESaq micropatterns onto the glass surface. (iii) A PDMS microfluidic device is then bonded to the patterned glass substrate to create (iv) a sealed microfluidic device encapsulating APTESaq micropatterns. (b) Lift-off nanocontact printing for APTESaq nanopatterns: (i) A plasma-activated NOA63 lift-off stamp was contacted with an APTESaq-inked flat PDMS stamp. (ii) The APTESaq patterned flat stamp was pressed onto a plasma activated glass slide for 5 s. (iii) A microfluidic channel was then bonded irreversibly to (iv) encapsulate the nanopatterns.

FIG. 2 Grafting of IgGs and DNA aptamers in APTESaq patterned microfluidic devices. (a) Biotinylated IgGs were grafted on the APTESaq pattern via EDC-NHS chemistry and labeled with fluorescent streptavidin dye to reveal an array of 100 μm squares within the microfluidic channels. (b) Alternatively, amine-terminated biotinylated aptamers were immobilized to the patterned APTESaq via BS3 chemistry and subsequently stained with streptavidin dye. Dotted lines depict microfluidic channel boundaries and scale bars are 200 μm. Illustrations portray the binding architecture of molecules within the patterns.

FIG. 3 Lift-off nanocontact printing for APTES nanopatterns. (a) SEM image depicting the nanoholes on the surface of an NOA63 lift off stamp. (b) Confocal microscopy image of fluorescently-labeled IgGs grafted onto the APTESaq nanopatterns within the microfluidic channels. The illustration depicts the binding architecture within patterns. Scale bars in (a) & (b) are 5 μm and 500 nm in the inset of (b).

FIG. 4 Aptamer and antibody-based immunoassay in APTESaq patterned devices. (a) Aptamer-based sandwich and (b) antibody-based immunoassays were carried out to detect IL6 and hCRP on aptamer-functionalized and antibody-functionalized APTESaq micropatterns respectively in microfluidic devices. (c) Histogram of normalized fluorescence intensity values is plotted for detection of 4 nM of hCRP versus that of blank and negative control (IL6). (d) Normalized fluorescence intensity values are plotted for the tested concentrations of hCRP. The solid line depicts the best curve fit and the dotted lines depict the limit of detection where the lowest concentration of hCRP detected was 4 nM. Scale bars are 100 μm for (a) & (c) and 200 μm for (b).

FIG. 5 APTESaq patterns in microfluidic devices are stable for 90 days at both 4° C. and room temperature. Fluorescently labeled IgGs were grafted on Day 1, Month 1 and Month 3 onto 6 different APTESaq-patterned microfluidic devices stored at 4° C. ((a), (b) & (c)) and at 25° C. ((d), (e) & (f)) respectively. (g) The histogram depicts the normalized fluorescence intensity, quantified on the APTESaq patterns for three tested conditions. Scale bars are 100 μm.

FIG. 6 Multi-protein patterning on APTESaq micro patterns using liquid dispensing robots and microfluidic devices. (a) Schematic illustrating a liquid dispensing robot delivering two different protein solutions to different portions of an APTESaq patterned substrate. (b) A liquid dispensing robot was used to locally deliver EDC-NHS activated Alexa-fluor 488 and 546-labelled fluorescent antibodies to different regions of an APTESaq array blocked with PEG-silane_{aq}. (c) Microcontact printing of an array of 100 μm wide APTESaq stripes was carried out prior to bonding of a microfluidic device containing channel arrays that were aligned perpendicular to each other. (d) Blocking was carried out using PEG-silaneaq prior to delivering solutions of fluorescently-labeled IgGs to alternating channels. After washing, alternating patterns of two fluorescently-labeled IgGs are patterned within the microfluidic channels. Scale bars are 2 mm in (b) and 300 μm in (d).

FIG. 7 Schematics of stamps for microcontact printing and microfluidic devices. Patterning stamp designs by AutoCAD (AutoDesk, USA) drawings: (a) 100 μm wide stripes with 100 μm spacing; (b) an array of 50 by 50 μm squares with 50 μm spacing. Microfluidic device designs by AutoCAD: (c) 100 μm wide and (d) 200 μm wide parallel channels with single inlet and single outlet, for unidirectional flows; and (e) 200 μm wide parallel channels connected with two different inlets, for opposite flow directions in alternating channels.

FIG. 8 Covalent patterning of biomolecules on surfaces using covalent microcontact printing. Conventional microcontact printing of fluorescently-labeled IgG in microfluidic channels yields (a) micrometer features prior to flow, (b) fluorescence in the regions of protein deposition diminishes due to flow in the microfluidic device and protein detachment. Covalent grafting of the proteins using covalent microcontact printing yields (c) micrometer features prior to flow, (d) fluorescence intensity remains the same under high flow rates and proteins remain bound to the surface. (e) Quantification of the fluorescence intensity verifies that proteins remain bound to the surface under flow conditions via the covalent grafting approach.

FIG. 9 Comparison of direct protein nanocontact printing and aqueous-based APTES nanocontact printing. (a) Conventional nanocontact printing was performed to directly pattern a glass substrate with nanodots of fluorescently-labelled Immunoglobulins (IgGs) (red) of 200 nm feature size, using the procedure previously described by Ricoult et al., (2013) [Ricoult, S. G., et al., Large Dynamic Range Digital Nanodot Gradients of Biomolecules Made by Low-Cost Nanocontact Printing for Cell Haptotaxis. Small, 2013. 9(19): p. 3308-3313.]. The nanopatterns of fluorescently labeled protein were imaged on an LSM 780 Confocal microscope (Zeiss, Japan). (b) A plasma-activated NOA63 lift-off stamp was contacted with an APTESaq-inked PDMS flat stamp. The APTESaq patterned flat stamp was pressed onto a plasma activated glass slide for 5 s. A microfluidic channel was then bonded irreversibly to encapsulate the nanopatterns. Fluorescence images reveal fluorescently-labeled IgGs grafted onto the APTESaq nanopatterns of 200 nm feature size within the microfluidic channels. These images demonstrate that the biomolecular nanopatterns generated via nanocontact printing of APTESaq followed by IgG grafting is similar to the nanopatterns created by direct nanocontact printing of IgGs. Scale bars are 6 μm in both (a) & (b).

FIG. 10 Antibody-based sandwich immunoassay to detect human C-reactive protein (hCRP).Microchannel (microfluidic channel boundaries indicated with dotted lines) substrates are first patterned with APTESaq patterns with capture antibodies grafted via BS3 chemistry. hCRP with concentrations varying from 2 nM to 217 nM are mixed with 1×PBS and flowed through the microfluidic device. Captured hCRP molecules were detected via a secondary antibody pair consisting of the same primary antibody and Alexa-fluor 546-labelled fluorescent secondary antibody. White squares in the images depict positive capture and detection of hCRP at varying concentrations, with fluorescence intensities proportional to the concentration of hCRP. Black squares depict unpatterned and blocked regions which serve as background. Scale bars are 200 μm.

### DESCRIPTION OF EMBODIMENTS

The invention is related to the area of micro- and nanocontact printing. In particular, it is related to micro- and nanocontact printing with aminosilanes: patterning surfaces of microfluidic devices for multiplexed bioassays.

In this work, we present a new means of creating micro- and nano-patterns of aminosilanes within microfluidic devices via an aqueous based microcontact printing technique. To minimize the diffusion of molecules into the PDMS stamp, we use water as the inking solvent and enforce short incubation and contact times during the printing process to preserve the pre-defined resolution of patterned features (H. Li, J. Zhang, X. Zhou, G. Lu, Z. Yin, G. Li, T. Wu, F. Boey, S. S. Venkatraman and H. Zhang, Langmuir, 2009, 26, 5603-5609.). These patterns then serve as the building block to couple multiple biomolecules in solution onto a single surface for subsequent bioassays. To validate the functionality of the coupled biomolecules, we carry out an aptamer based immunoassay to detect Interleukin 6 (IL6) and an antibody based immunoassay for the detection of human C-reactive protein (hCRP). We probe the stability of APTES patterns and demonstrate the possibility of fabricating pre-stored and ready-to-use bioassay devices with a shelf life of at least 3 months. Finally, we verify the multiplexing capability on a single patterned surface by delivering different biomolecules to different regions of the patterned array with the help of microfluidic networks and liquid dispensing technologies.

Before the present invention is described in detail, it is to be understood that this invention is not limited to the particular methodology, devices, solutions, arrays, kits, substrates or apparatuses described, as such methodology, devices, solutions, arrays, kits, substrates or apparatuses can, of course, vary. It is also to be understood that the terminology used herein is for the purpose of describing particular embodiments only, and is not intended to limit the scope of the present invention.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

Definitions

The term “biomolecule” is used herein to refer to any chemical or biochemical structure present in living things which includes, but is not limited to, nucleotides, peptides, antibodies, carbohydrates and lipids.

The term “array” is used herein to refer to proteins, peptides, antibodies, nucleic acids, carbohydrates and lipids microarrays. Specific proteins, peptides, antibodies, nucleic acids, carbohydrates and lipids can be immobilized on solid surfaces to form arrays.

The term “binding” is used herein to refer to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, peptides, antibodies, nucleic acids, carbohydrates and lipids. Polypeptides that form stable complexes with other molecules are often referred to as receptors while their binding partners are called ligands. Polynucleotides can also form stable complex with themselves or others, for example, DNA-protein complex, DNA-DNA complex, DNA-RNA complex.

The term “peptides” is used herein to refer to proteins, fragments of proteins, and peptides, whether isolated from natural sources, produced by recombinant techniques, or chemically synthesized.

The term “nucleic acids” is used herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

The term “probe” is used herein to refer to a structure comprising nucleic acids, as defined above that contains a nucleic acid sequence that can bind to a corresponding target. The nucleic acids regions of probes may be composed of DNA, and/or RNA, and/or synthetic nucleotide analogs. “Probe” is also used herein to refer to a structure comprising proteins, peptides, antibodies, carbohydrates and lipids that can bind to a corresponding target.

The term “nano features” is used herein to refer to predefined depositions of a given material of biomolecule or silane where the size of the depositions is inferior to 1000 nm and where the structures are predefined such as nanodots, nanoposts, and nanoislands.

“Silane” herein refers to silane compounds such as, not limited to, 3-aminopropyltriethoxysilane(APTES), triethoxysilypropyl succinic anhydride (TESPSA), (3-Glycidyloxypropyl)trimethoxysilane (GPTMS), octadecyltrichlorosilane (OTS), trichloro(1H, 1H, 2H 2H-perfluorooctyl) silane, trichlorosilanes, methyltrimethoxysilane, methyltriethoxysilane, phenyltrimethoxysilane, phenyltriethoxysilane, dimethyldimethoxysilane, dimethyldiethoxysilane, diphenyldimethoxysilane, diphenyldiethoxysilane, diphenyldiethoxysilane, tetramethoxysilane, tetraethoxysilane and the like. 3-aminopropyltriethoxysilane (APTES) and silane terminated with amine, carboxylate or thiol groups are preferable.

“Multiplexing” or “a multiplexed bioassay” herein refers to an assay or other analytical method in which the presence of multiple target molecules can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime).

It is understood that aspects and embodiments of the invention described herein include “consisting” and/or “consisting essentially of aspects and embodiments.

Other objects, advantages and features of the present invention will become apparent from the following specification taken in conjunction with the accompanying drawings.

In a high-throughput manner, microarray technologies enable the evaluation of up to tens of thousands of molecular interactions simultaneously. Microarrays have made significant impact on biology, medicine, drug discovery. DNA microarray-based assays have been widely used, including the applications for gene expression analysis, genotyping for mutations, single nucleotide polymorphisms (SNPs), and short tandem repeats (STRs). And polypeptide and chemical microarrays have emerged as two important tools in the field of proteomics. Chemical microarray, a form of combinatorial libraries, can also be used for lead identification, as well as optimization of these leads. In this era of bioterrorism, the development of a microarray capable of detecting a multitude of biological or chemical agents in the environment will be of great interest to the law enforcement agencies.

According to some embodiments of the present invention, assay methods, a substrate, an array, a kit for analysis of molecular interactions are provided. The inventive technology improves specificity and sensitivity of microarray- based assays while reducing the cost of performing genetic assays.

Methods

1. Patterning and Fabrication Procedures of the Present Invention

1-1. Soft Lithography

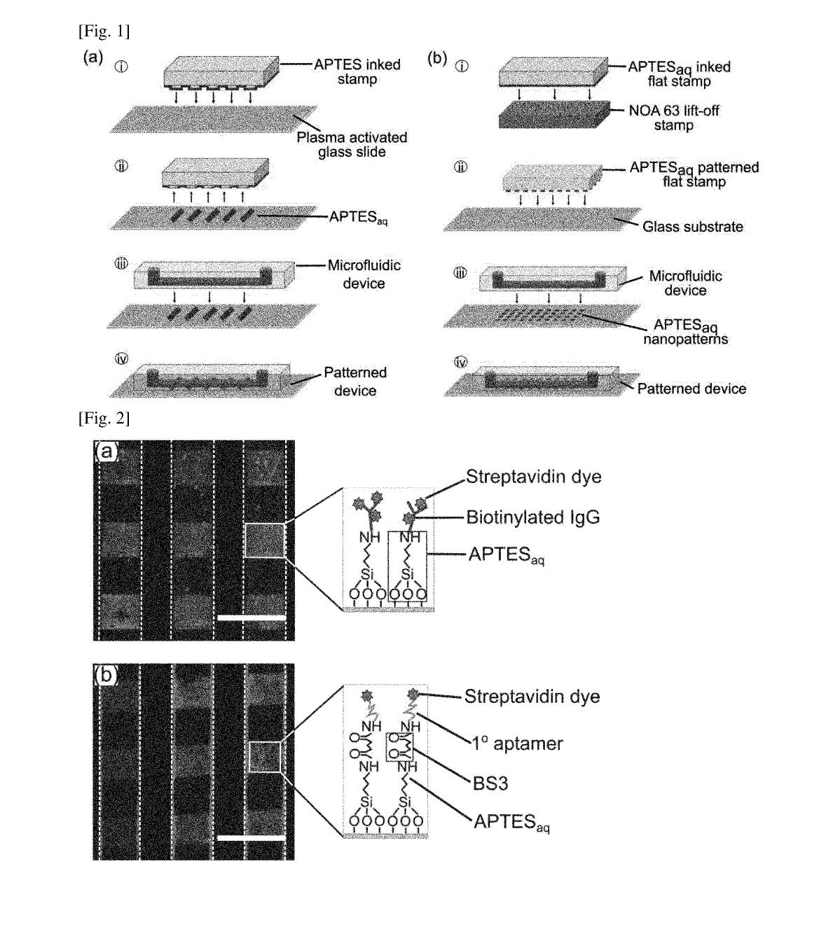

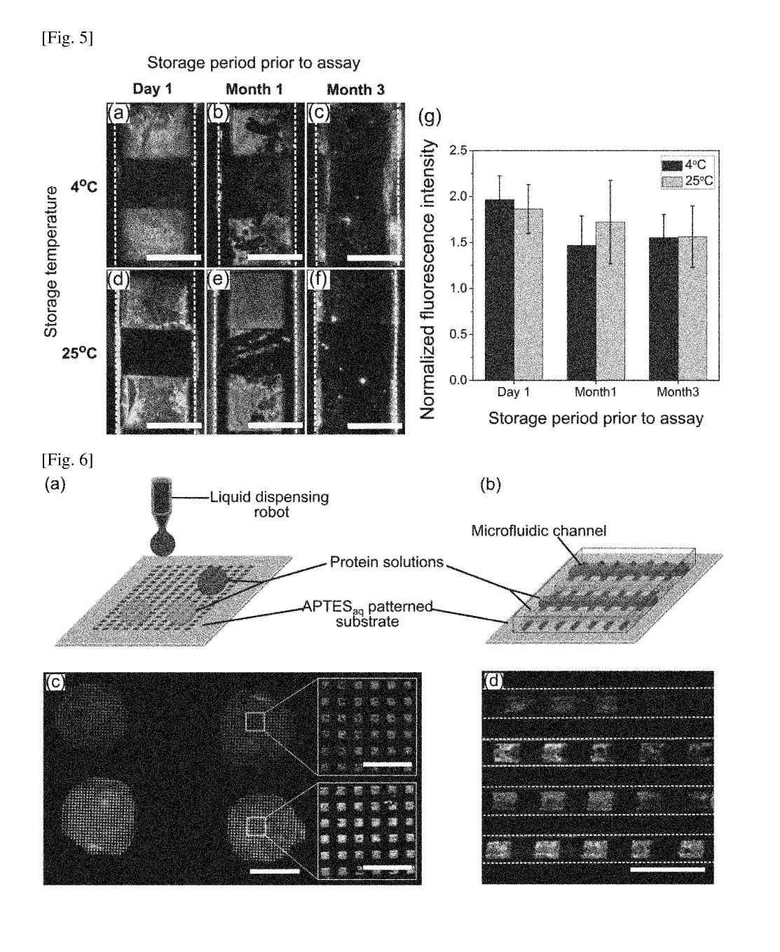

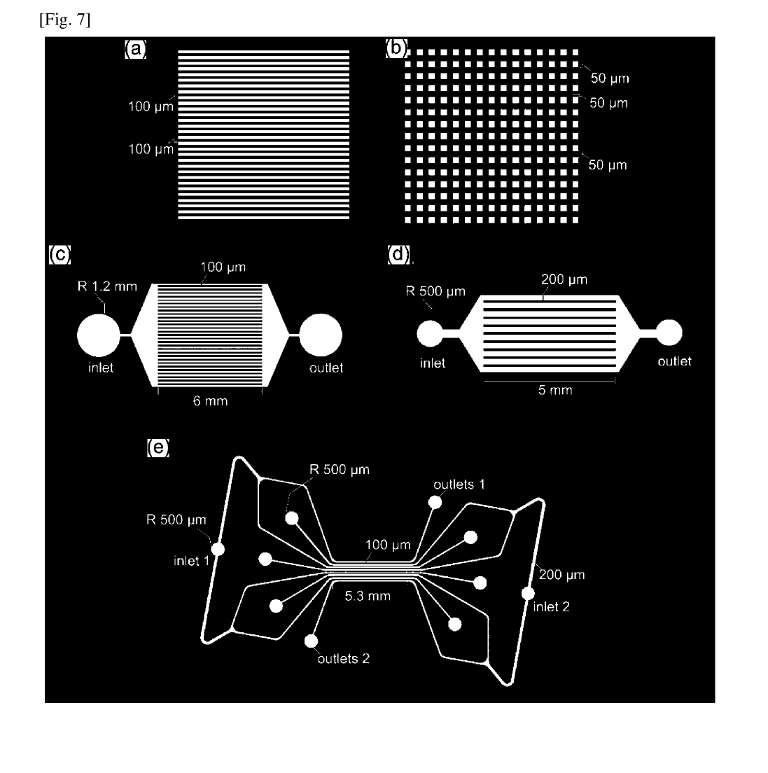

Stamps and microfluidic devices can be designed with AutoCAD (AutoDesk, USA). Stamp designs can comprise, but not limited to, (i) 100 μm wide stripes with 100 μm spacing (schematic in FIG. 1a(i)), and (ii) an array of 50 by 50 μm squares (FIG. 6a-b) separated by 50 μm. Three different designs of microfluidic devices were designed in AutoCAD: (i) 100 μm wide and (ii) 200 μm wide parallel channels with single inlet, for unidirectional flow; and (iii) 200 μm wide parallel channels with two different inlets, for opposite flow directions in alternating channels. More detailed schematics are illustrated in FIG. 7. For fabricating the master for the devices and stamps, silicon wafers (such as 4-inch in diameter, E&M Corp. Ltd., Japan) were coated with a 75 μm layer of a photosensitive polymer of photoresist (mr-DWL 40 photoresist; Microresist technologies, Germany), the thickness the layer of a photosensitive polymer can be defined by the photoresist and it can be varied from 700 nm to 200 μm, and the features were patterned by photolithography using a device such as a DL1000 maskless writer (NanoSystem Solutions, Japan) and developed by using a developer such as mr-Dev 600 developer (Microresist Technologies, Germany). After thorough baking and cleaning, the wafers were coated with an antiadhesive layer by exposing it to silane such as trichloro(1H, 1H, 2H 2H-perfluorooctyl) silane (Sigma-Aldrich, Japan) in vapor phase in a desiccator. Microfluidic devices and stamps with the inverse copy of the pattern present on the Si-wafer were obtained by pouring 10:1 poly-(dimethylsiloxane) (PDMS) (DOW Corning, Japan) on the wafer and curing the pre-polymer for 15 h to 48 h, preferably for 24 h at 60° C. after degassing to remove air bubbles. For the lift-off nanocontact printing process, flat PDMS stamps were fabricated by polymerizing the aforementioned mixture on a blank Si-wafer.

1-2. Microcontact Printing (μCP) in Microfluidic Devices

As illustrated in the schematic in FIG. 1a, the patterned stamps can be inked with 10 μL of 1% aqueous APTES (APTESaq) by volume for 1-3 min under a plasma activated coverslip. We can also use mercaptopropyltriethoxysilane, Octadecy trichlorosilane (ODTCS), triethoxysilypropyl succinic anhydride (TESPSA), (3-Glycidyloxypropyl)trimethoxysilane (GPTMS), octadecyltrichlorosilane (OTS) and the like instead of APTES. The stamps can be rinsed with milli-Q water (Millipore, Japan) for 10 s before rapid drying with a strong pulse of N₂ gas. The inked PDMS stamp is then contacted with a plasma activated (Harrick Plasma, USA) glass slide for 5 s. The prepared plasma-activated PDMS microfluidic device is bonded, perpendicular to the printed substrate. The assembled device is then heated at 85° C. for 5 minutes on a hot plate to simultaneously ensure covalent reaction of APTESaq and to irreversibly bond the microfluidic device to the substrate. The substrates can be made of glass, silicon, or plastic, preferably glass or plastic, more preferably glass.

1-3. Nanopatterned Lift-Off Stamps

Nanopatterned (nano feature is pattered from 10 nm to 1000 nm, preferably 20 nm to 800 nm, more preferably 50 nm to 500 nm) PDMS replicas are fabricated using the protocol previously described by Ricoult, et al. (S. G. Ricoult, M. Pla-Roca, R. Safavieh, G. M. Lopez-Ayon, P. Grutter, T. E. Kennedy and D. Juncker, Small, 2013, 9, 3308-3313). Briefly, nanopatterns consisting of a square array (200 nm in length, 200 nm in width, with 2 μm in spacing) are first created using Clewin Pro 4.0 (Wieweb software, Hengelo, Netherlands). A 4-inch silicon wafer is coated with PMMA resist and the dot arrays are patterned by electron beam lithography (VB6 UHR EWF, Vistec), followed by 100 nm reactive ion etching (System100 ICP380, Plasmalab) into the Si. After cleaning, the wafer is coated with an anti-adhesive layer by exposing it to perfluorooctyltriethoxysilane (Sigma-Aldrich, Oakville, ON, Canada) in vapor phase in a desiccator. An inverse polymer copy of the Si wafer is obtained after curing PDMS on the patterned wafer as described in the previous section to generate nanopillars. The lift-off stamp consisting of nanoholes with an inverse copy of the PDMS master (FIG. 3a) is finally obtained by curing Norland Optical Adhesive 63 (NOA63, Norland Products, Cranbury, N.J.) on the PDMS stamp with 600 W of UV light for 40 seconds in a Uvitron 600W UVA Enhanced Lamp (310-400 nm; 100% intensity) (Uvitron International, Inc., West Springfield, Mass.).

1-4. Lift-Off Nanocontact Printing

A flat PDMS stamp is inked for 1-3 min with the 1% silane such as APTESaq solution as mentioned above (FIG. 1b). The concentration of the silane solution can be 0.5% to 5%. After rinsing with Milli-Q water for 10 s, the inked stamps are briefly dried under a stream of N₂ and immediately brought into contact with a plasma activated a photosensitive polymer (such as NOA63) lift-off stamp for 5 s. The PDMS is separated from the photosensitive polymer (such as NOA63) lift-off stamp and the APTESaq in the contact area are transferred to the NOA63 lift-off stamp, while the remaining APTESaq molecules are transferred to the final substrate by printing the PDMS stamp for 5 s onto a plasma activated glass surface.

2. APTESaq-Biomolecule Grafting Within Patterned Devices

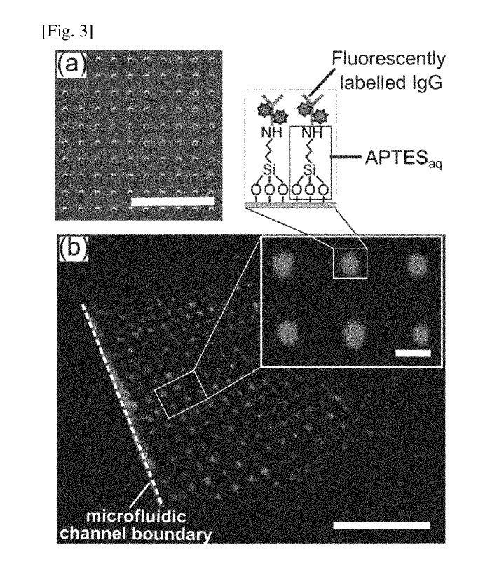

Following APTESaq patterning and device assembly, the unpatterned regions within the device were blocked for 30 min by flowing a solution of 2 wt % PEG-silaneaq through the device. The concentration of the PEG-silane_{aq} can be 1% to 5%. Fluorescently labelled Immunoglobulins or protein of interest at 10 μg/ml were covalently grafted on the APTESaq patterned surface by employing EDC-NHS chemistry at a 10-fold molar excess of EDC (2 μM) and NHS (5 μM) to protein (see FIGS. 2a & b). DNA aptamers were grafted using BS3 (bis(sulfosuccinimidyl)suberate) chemistry where 100 μM of BS3 in Milli-Q water was flowed over the patterns in the device to enable reaction with the available terminal amine (—NH2) group on the APTESaq, followed by the aptamer solution (10 μg, g/ml) in 20 mM HEPES buffer and 50 mM of Glycineaq to quench unreacted BS3. Unreacted components were washed with wash buffer (0.05% Tween 20 in 1×PBS) following each step of the reaction.

3. Imaging and Analysis

NOA63 lift-off stamps were imaged using Quanta 250 FEG scanning electron microscope (FEI, Japan) at 5 kV with a spot size of 3.5 using an ETD Detector to detect secondary electrons. Micro- and nanopatterns of fluorescently labeled protein were imaged on a Ti-E Eclipse inverted fluorescent microscope (Nikon, Japan) and an LSM 780 Confocal microscope (Zeiss, Japan). All images were captured with fixed exposure times within each experiment, which varied from 1 to 10 s for all the images shown in this work. Mean fluorescence intensity measurements were obtained by performing image analysis in ImageJ (NIH, USA). Images were processed post quantification to increase the contrast through linear modifications in ImageJ.

Arrays

A microarray is a multiplex technology widely used in molecular biology and medicine. Microarrays can be fabricated using a variety of technologies, including printing with fine-pointed pins, photolithography using pre-made masks, photolithography using dynamic micromirror devices, inkjet printing, microcontact printing, or electrochemistry on microelectrode arrays. In standard microarrays, the probe molecules are attached via surface engineering to a solid surface of supporting materials, which include glass, silicon, plastic, hydrogels, agaroses, nitrocellulose and nylon.

The systems described herein may comprise two or more probes that detect the same target biomolecules. For example, in some embodiments where the system is a microarray, the probes may be present in multiple (such as any of 2, 3, 4, 5, 6, 7, or more) copies on the microarray. In some embodiments, the system comprises different probes that detect the same target biomolecules. For example, these probes may bind to different (overlapping or non-overlapping) regions of the target biomolecules.

Any probes that are capable of determining the levels of target biomolecules can be used. In some embodiments, the probe may be an oligonucleotide (nucleic acids), peptides, antibodies, carbohydrates or lipids. It is understood that, for detection of target biomolecules, certain sequence or structure variations are acceptable. In some embodiments, the probe comprises a portion for detecting the target biomolecules and another portion. Such other portion may be used, for example, for attaching the biomolecules to a substrate. In some embodiments, the other portion comprises a non-specific sequence or nonspecific structure for increasing the distance between the complementary structure portion and the surface of the substrate.

The present invention provides an array of biomolecules comprising a substrate and a probe molecule, wherein the surface of the substrate has patterned nano features of silane. The unpatterned surface of the substrate is preferably blocked with PEG-silane. In addition, the diameter of the nano features is patterned from 10 nm to 1000 nm, preferably 20 nm to 800 nm, more preferably 50 nm to 500 nm. The probe molecule is selected from the group consisting of proteins, peptides, antibodies, nucleic acids, carbohydrates and lipids. The probe molecule is conjugated onto nano features of silane on the substrate. The method section can be referred for details of the Arrays of the present invention.

The assays based on the arrays of the present invention may be implemented in a multiplex format. Multiplex methods are provided employing 2, 3, 4, 5, 10, 15, 20, 25, 50, 100, 200, 500, 1000 or more different capture probes which can be used simultaneously to assay for amplification products from corresponding different target polynucleotides. Methods amenable to multiplexing, such as those taught herein, allow acquisition of greater amounts of information from smaller specimens. The need for smaller specimens increases the ability of an investigator to obtain samples from a larger number of individuals in a population to validate a new assay or simply to acquire data, as less invasive techniques are needed.

Kits

The present invention provides a kit comprising a substrate and a probe molecule, wherein the surface of the substrate has patterned nano features of silane. The unpatterned surface of the substrate is preferably blocked with PEG-silane. In addition, from 10 nm to 1000 nm, preferably 20 nm to 800 nm, more preferably 50 nm to 500 nm. The probe molecule is selected from the group consisting of proteins, peptides, antibodies, nucleic acids, carbohydrates and lipids. The method section can be referred for details of the Kits of the present invention.

Substrates

The present invention provides a substrate for an array of biomolecules, wherein the surface of the substrate has patterned nano features of silane for conjugating a probe molecule. The substrates can be made of glass, silicon, or plastic, preferably glass or plastic, more preferably glass. The unpatterned surface of the substrate is preferably blocked with PEG-silane. In addition, from 10 nm to 1000 nm, preferably 20 nm to 800 nm, more preferably 50 nm to 500 nm. The probe molecule is selected from the group consisting of proteins, peptides, antibodies, nucleic acids, carbohydrates and lipids. The method section can be referred for details of the Substrates of the present invention.

### EXAMPLE 1

APTESaq Micropatterns for Grafting of Biomolecules Within Microfluidic Devices

To facilitate the patterning of biomolecules within microfluidic devices regardless of the molecular charge, a microcontact printing process was developed to print aqueous APTES (APTESaq) in closed microfluidic devices (schematic in FIG. 1a), to create covalent bonds between the surface and the biomolecules. Here, APTESaq was printed onto a plasma activated glass slide using a PDMS stamp (FIG. 1a(i)-(ii)) and bonded to a plasma activated PDMS microfluidic device to enclose the vertical print within the horizontal microfluidic channels. The assembled device was heated at 85° C. for 5 minutes to drive the formation of a covalent siloxane bond between the reactive silanols in APTES and the hydroxyl (—OH) groups on the glass substrate (J. A. Howarter and J. P. Youngblood, Langmuir, 2006, 22, 11142-11147.). The unpatterned regions within the device were then blocked for 30 min by flowing a solution of 2% PEG-silaneaq through the device. PEG-silaneaq not only acts as a non-biofouling agent but also prevents diffusion of APTESaq on the patterned substrate. A mixture of the desired biomolecules and appropriate linkers were then flowed through the patterned channels of the device to allow reaction and covalent grafting to the APTESaq.

FIGS. 2a & b depict grafting of biotinylated Immunoglobulins (IgGs) via EDC-NHS chemistry and amine (—NH₂)-terminated biotinylated aptamers using BS3 (bis(sulfosuccinimidyl)suberate), an —NH₂ to —NH₂ linker, onto APTES patterns respectively. These patterned biotinylated molecules were then subsequently labeled with fluorescent streptavidin dye to reveal their successful covalent grafting as red squares within the microfluidic channels. Additionally, the integrity of the biomolecule patterns under high shear stresses in the microfluidic channels was confirmed as shown in Supplementary FIG. 8.

In previously described methods (G. Arslan, M. Ozmen, I. Hatay, I. H. Gubbuk and M. Ersoz, Turkish Journal of Chemistry, 2008, 32, 313-321.), when the silanes were inked with toluene, there is a high probability of PDMS swelling and subsequent change in feature sizes.

Additionally, silane-toluene reservoirs are created within the stamp, which diffuse out of the stamp when printed onto glass surfaces for long contact times, 35 ultimately leading to loss of resolution. With our protocol, by using water as the inking solvent, we not only limit the probability of silane reservoir formation and swelling of the stamp, but also reduce leakage upon contact by using combined inking and printing times on the order of a few minutes. Thus, the desired patterning dimension is maintained during and after the printing process. These results depict the compatibility of this technique with glass-based microfluidic devices to covalently pattern not only proteins but also effectively couple other biomolecules such as DNA aptamers, carbohydrates, and lipids onto their substrates for subsequent bioassay applications.

### EXAMPLE 2

Aminosilane Nanopatterns for Grafting of Biomolecules Within Microfluidic Devices

In addition to successfully creating micropatterns of APTES to covalently pattern biomolecules, we further demonstrate a simple lift-off nanocontact printing method for creating nanopatterns of APTESaq within microfluidic channels to subsequently graft biomolecules covalently. Following a previous protocol (S. G. Ricoult, M. Pla-Roca, R. Safavieh, G. M. Lopez-Ayon, P. Grutter, T. E. Kennedy and D. Juncker, Small, 2013, 9, 3308-3313), disposable epoxy lift-off stamps (FIG. 3a) replicating a wafer with nanoholes were obtained through a double replication process via a PDMS intermediate replica.

A flat PDMS stamp was then inked with the APTESaq solution, rinsed and dried before being pressed against the plasma activated lift-off stamp for 5 s (FIG. 1b(i)). The flat PDMS stamp with the remaining APTESaq nanopattern was then pressed against a plasma activated glass slide for 5 s (FIG. 1b(ii)). By utilizing a flat PDMS stamp to pattern nanoscale features of APTES, we reduce the risk of nanopillar collapse and subsequent smudging of features, a common hurdle while using nanopillar PDMS stamps. Finally, a plasma activated microfluidic device was bonded onto the printed substrate, heated at 85° C. immediately after printing (FIG. 1b(iii)-(iv)) and blocked with PEG-silaneaq to reduce non-specific adsorption. Subsequently, fluorescently-labeled IgGs were grafted onto patterned APTESaq via EDC-NHS chemistry to reveal 200 nm nanodots of proteins (FIG. 3b).

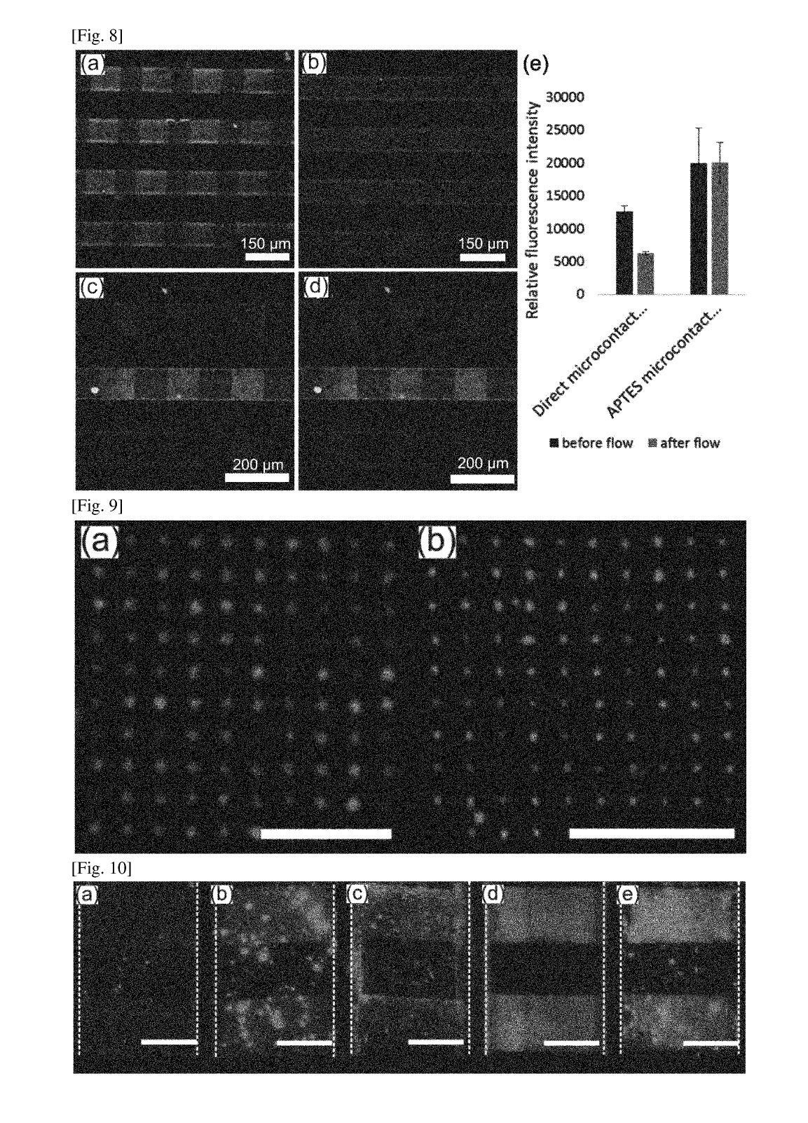

Several prior studies have reported the potential of nanocontact printing to satisfy the growing demand of creating biomolecule nanopatterns to achieve single molecule detection (B. R. Takulapalli, M. E. Morrison, J. Gu and P. Zhang, Nanotechnology, 2011, 22, 285302.; H.-W. Li, B. V. Muir, G. Fichet and W. T. Huck, Langmuir, 2003, 19, 1963-1965.; J. Gu, X. Xiao, B. R. Takulapalli, M. E. Morrison, P. Zhang and F. Zenhausern, Journal of Vacuum Science & Technology B, 2008, 26, 1860-1865.). However, few reports have incorporated these patterns into microdevices for subsequent microfluidic bioassays (A. S. Andersen, W. Zheng, D. S. Sutherland and X. Jiang, Lab on a Chip, 2015, 15, 4524-4532.). Additionally, the reliance of the patterning processes on physisorption proves to be a drawback as the biomolecules are susceptible to detachment from the surface due to the presence of high shear stress introduced by the flow. Our new protocol here addresses all these challenges: by employing lift-off nanocontact printing of APTESaq on glass substrates, covalently tethered nanopatterns of proteins with a resolution of 200 nm can be easily integrated into microfluidic devices. Additionally, these protein nanopatterns are created with the same efficiency as the previously described direct nanocontact printing approach by Ricoult, et al. (S. G. Ricoult, M. Pla-Roca, R. Safavieh, G. M. Lopez-Ayon, P. Grutter, T. E. Kennedy and D. Juncker, Small, 2013, 9, 3308-3313.), see more details in FIG. 9.

### EXAMPLE 3

Aptamer-Based and Antibody-Based Immunoassays

Interleukin-6 (IL6) (J. S. Yudkin, M. Kumari, S. E. Humphries and V. Mohamed-Ali, Atherosclerosis, 2000, 148, 209-214.; A. G. Vos, N. S. Idris, R. E. Barth, K. Klipstein-Grobusch and D. E. Grobbee, PloS one, 2016, 11, e0147484.) and human C-reactive protein (hCRP) (I. Kushner, Science, 2002, 297, 520-521.; P. M. Ridker, Circulation, 2003, 107, 363-369.) are the most important biomarkers of neurological, cardiovascular and other pathophysiological conditions that arise from tissue inflammation or infection. Quantitative detection of these biomarkers has immensely helped in early diagnosis and treatment of these diseases. In order to accurately diagnose these diseases, sensitive assays and biosensing technologies are required to reliably detect minute quantities of these biomarkers (S. K. Vashist, A. Venkatesh, E. M. Schneider, C. Beaudoin, P. B. Luppa and J. H. Luong, Biotechnology advances, 2016, 34, 272-290.; A. Qureshi, Y. Gurbuz and J. H. Niazi, Sensors and Actuators B: Chemical, 2012, 171, 62-76.). Therefore, to test the sensitivity and biofunctionality of our APTESaq-micropatterned microfluidic devices, sandwich-based immunoassays were carried out to qualitatively and quantitatively detect IL6 and hCRP with the help of either aptamers or antibodies respectively.

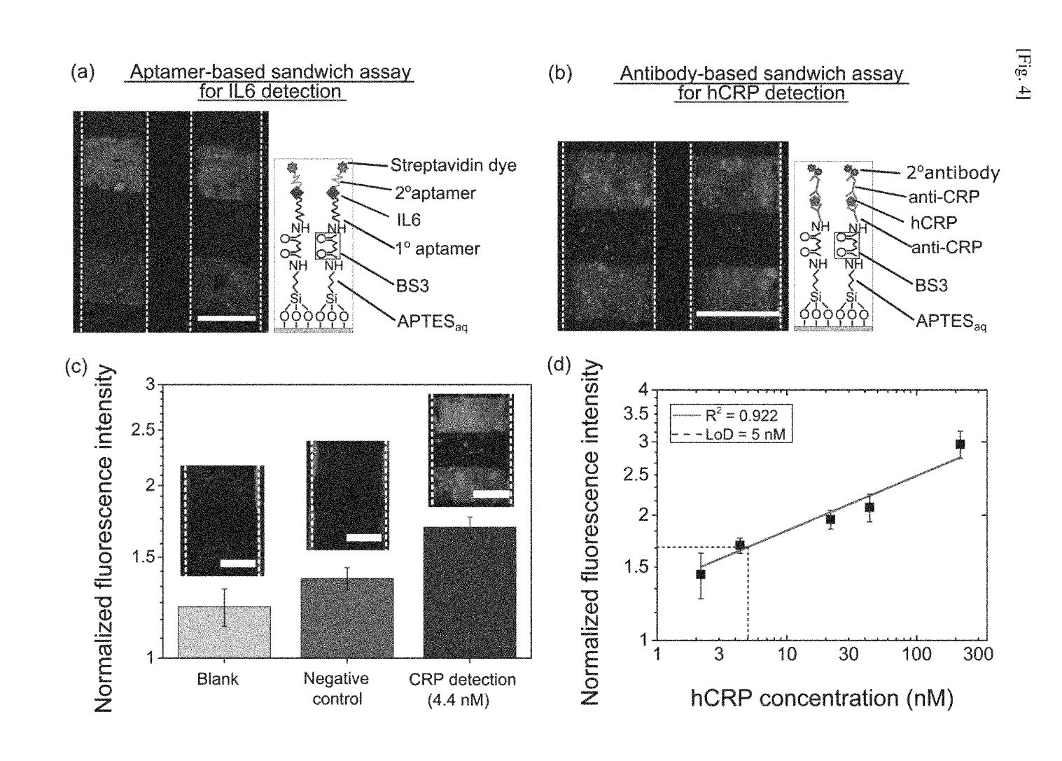

For the aptamer-based immunoassay, —NH₂-terminated aptamers specific to IL6 were grafted onto the APTESaq micropatterns via BS3 chemistry after blocking. Subsequently, 470 nM of IL6 was detected with the help of a complimentary biotinylated detection aptamer and streptavidin dye (see FIG. 4a). To carry out an antibody-based sandwich immunoassay to detect hCRP, capture antibodies against hCRP were grafted onto APTESaq patterns via BS3 chemistry. These antibodies served to capture 217 nM of hCRP when detected by a detection antibody pair that consisted of the same capture antibodies and complimentary Alexa-fluor 546-labelled fluorescent secondary antibodies, see detailed schematic in FIG. 4b. Given the pentameric structure of hCRP, the same capture antibody was used as the detection antibody at a concentration of 10 μg/ml.

To further characterize the sensitivity of these patterned devices, we focused on the antibody-based sandwich immunoassay. Varying concentrations from 2 nM to 217 nM of hCRP mixed in PBS were flowed through microchannels patterned with capture antibodies against hCRP grafted onto APTESaq via BS3 chemistry. A range of concentrations of hCRP from 4-200 nM was successfully detected via the detection antibody pair and qualitatively analyzed by fluorescence microscopy (more details are shown in Figure S4a-e in the SI document).

To estimate the detection of hCRP quantitatively, the normalized fluorescence intensity was calculated for each condition by measuring the ratio of mean pixel intensity of the patterned region (red) to that of the unpatterned region (black), averaged over 3 images each with 9 patterned squares in each image. A blank reaction was carried out by flowing the detection antibody pair over the grafted capture antibody to account for the nonspecific adsorption. The histogram in FIG. 4c depicts the normalized fluorescence intensity plotted for positive detection of the lowest detectable concentration of 4.4 nM of hCRP versus that of the blank reaction and a negative control (i.e., IL6 flowed through the anti-hCRP grafted microchannels). The high levels of nonspecific adsorption could be caused by the use of the same primary antibody as both the capture and detection antibodies.

FIG. 4d displays the normalized fluorescence intensity values captured for each concentration of hCRP detected (in black squares), while the solid line is the best linear curve fit (B. Nix and D. Wild, The immunoassay handbook, 2001, 2, 198-210.) with an R2 value of 0.922. Results illustrate that the lowest detectable concentration of hCRP in these patterned devices was 4 nM as estimated by the limit of detection calculated to be 1.67 relative fluorescence units (RFU), derived from the following formula (D. A. Armbruster and T. Pry, Clin Biochem Rev, 2008, 29, S49-52.):

LoB=Meanblank+1.645(SDblank), (1)

LoD=LoB+1.645(SDlcs) (2)

where LoB, SD, LoD and lcs are the limit of blank, standard of deviation, limit of detection and lowest concentration sample respectively.

The successful detection of clinically significant levels of IL6 and hCRP validates the biofunctionality of these patterned devices. The sensitivity of these devices can be significantly improved in the future with more specific aptamer or antibody combinations coupled with label-free detection systems.

### EXAMPLE 4

Stability of Aminosilane Patterns

To assess the stability of the APTESaq patterns, microfluidic device substrates were pre-patterned with APTESaq by microcontact printing perpendicular to the microfluidic channels, in stripes of 100 μm separated by 100 μm in spacing. Thirty patterned and sealed devices were stored in plastic containers after blocking with PEG-silaneaq for up to 3 months at room temperature (25° C.) or at 4° C. in the absence of vacuum. Three devices per testing condition were characterized to determine the efficiency of grafting of fluorescently labeled IgGs (Immunoglobulins) on the APTESaq patterns.

Square fluorescent bands shown in FIGS. 5a-f illustrate successful grafting of the IgGs. To quantify the efficiency of grafting, fluorescent squares were considered as signal and unpatterned regions as background. Normalized fluorescence intensity values were quantified by ImageJ using the same method of analysis as previously described, and plotted for each of the testing conditions (3 devices per condition) (FIG. 5g), where the standard deviations indicate averaging uncertainty. FIG. 5 demonstrates that the APTESaq is stable for 3 months when stored at either 4° C. or 25° C. and can be used to graft biomolecules prior to immunoassay to eliminate the concern of biodegradation arising from the storage of patterned biomolecules.

It is worth noting that the initially microcontact printed APTESaq patterns already have a small level of inhomogeneity as seen in FIGS. 5a & d. This may be due to the oligomerization of highly reactive APTESaq molecules in water that form aggregates when inked on the stamp before the printing process (G. C. Allen, F. Sorbello, C. Altavilla, A. Castorina and E. Ciliberto, Thin Solid Films, 2005, 483, 306-311.; E. T. Vandenberg, L. Bertilsson, B. Liedberg, K. Uvdal, R. Erlandsson, H. Elwing and I. Lundstrom, Journal of Colloid and Interface Science, 1991, 147, 103-118.). This can be reduced by preparing fresh aqueous APTESaq solution prior to the inking process, reducing inking times and eliminating the step where the inked stamp is rinsed with water prior to print. Additionally, although the presence of fluorescently-labelled IgGs on devices stored for 3 months depicts presence of APTESaq (FIGS. 5c & f), gradual degradation of APTESaq is seen with time. As elucidated in previous literature (P. M. S. John and H. Craighead, Applied physics letters, 1996, 68, 1022-1024.) this degradation could be either owing to (i) moisture aided decomposition of APTESaq (N. A. Lapin and Y. J. Chabal, The Journal of Physical ChemistryB, 2009, 113, 8776-8783.) due to the high humidity environment present while performing the experiments, (ii) gradual self —NH₂-catalyzed hydrolysis and removal of the covalent siloxane of APTESaq (J. A. Howarter and J. P. Youngblood, Langmuir, 2006, 22, 11142-11147.), or (iii) incomplete covalent binding of APTESaq to the glass substrate (R. M. Pasternack, S. Rivillon Amy and Y. J. Chabal, Langmuir, 2008, 24, 12963-12971).

By printing aminosilanes that are insensitive to enzymes and subsequently capturing the biomolecules at the time of the bioassay, we highlight the following advantages: 1) the patterned substrates can be stored on the order of months before carrying out the bioassay, 2) biomolecules are less likely to be affected by denaturation associated with external stresses since they are delivered in solution, and 3) interaction sites can be accurately engineered by precisely designing the silanes and biomolecules thereby providing control over the orientation of the biomolecules. Additional experiments are being carried out to further probe and improve the chemical viability of APTESaq patterns on substrates upon storage, which will be reported in the future.

### EXAMPLE 5

Multiplexing on Aminosilane Patterned Substrates

To overcome the one stamp-one ink characteristic of microcontact printing, we use APTESaq patterns to capture and covalently graft different locally delivered biomolecules. To visually demonstrate the capability of patterning multiple biomolecules onto a single surface, two different solutions of EDC-NHS activated Alexa-fluor 488 and 546-labelled fluorescent antibodies were delivered onto the patterned substrate by two modes of liquid delivery. First, an array of squares (50 by 50 μm) of APTESaq was patterned on a plasma activated glass slide by microcontact printing and blocked with 2% PEG-silaneaq. Liquid dispensing robots (Musashi Engineering, Japan) were then used to deliver microliter volumes of droplets containing the two protein solutions (FIG. 6a) to achieve a microarray composed of multiple biomolecules on the patterned surface (FIG. 6b).

Alternatively, microfluidic devices (FIG. 6c) with channel arrays were bonded on an array of 100 μm wide APTESaq stripes aligned perpendicular to the direction of the microfluidic channels. After blocking with PEG-silaneaq, the two solutions of EDC-NHS activated fluorescently-labeled antibodies were fed into the channels and covalently grafted onto the APTESaq patterns within the channels (FIG. 6d).

One of the major obstacles in achieving multipatterning by microcontact printing has been the necessity of fabrication of complex stamps that either contained microfluidic circuits or gradient generators on the stamp to create patterned concentration gradients on substrates. In comparison, the aminosilane printing approach coupled with microfluidics introduced in this work, facilitates the creation of large and stable arrays composed of multiple biomolecules presented via covalent bonds in a single device. By making use of the localized delivery available in microfluidic devices or liquid dispensing platforms, multi-protein patterns could easily be achieved within a single array. Additionally, with the advent of nanofluidic devices and liquid dispensing robots delivering picolitre droplets, densely packed nanoarrays can undoubtedly be achieved in the near future.

To create biomolecular patterns within microfluidic channels, we introduced a micro- and nanocontact printing method to pattern amino terminated silanes on a desired planar surface, with feature sizes ranging from a few hundred microns down to 200 nm. This protocol provides several key advantages. First, owing to its compatibility with PDMS, water can be used as the inking solvent to pattern APTES onto glass substrates. Next, the microfluidic channels deliver a blocking solution, to (i) limit the diffusion of volatile silanes as well as (ii) inhibit biofouling. Micro- and nanopatterns can be grafted with different biomolecules such as proteins and DNA in controlled orientations for subsequent immunoassay applications within these devices. Additionally, the APTESaq patterns maintain their ability to covalently graft biomolecules to the surface for at least 3 months after printing with no significant difference between storage conditions at room temperature or at 4° C., thereby demonstrating their storage potentials. By grafting biomolecules onto pre-patterned substrates prior to use, it greatly preserves the functionalities of the grafted biomolecules with minimized risks of biodegradation, accompanied by simplified operation protocols.

By demonstrating successful DNA-based immunoassays and antibody-based immunoassays carried out on microcontact printed aminosilane patterns, we demonstrated the biofunctionality of these prints areas thereby describing the overall potential of this technology in the field of bioassay applications. To demonstrate the multiplexing potentials of this technology, localized delivery available in microfluidic devices or liquid dispensing platforms were used to achieve multi-protein patterning within a single array.

Applications for patterned surfaces are broad, but their translation from the lab to commercial products has been hindered by limited abilities to integrate the patterns into microfluidic devices with control. With this simple patterning technique, it could help in accelerating the translation of these patterned substrates from the lab to commercial products for the development of integrated bioassays suitable for commercialization in the near future.

Reagents and Materials

(3-Aminopropyl) triethoxysilane (APTES) and 2-methoxy (polyethyleneoxy) 6-9 propyl tricholoro silane (PEG-silane) were purchased from Nacalai, Japan. 1-ethyl-3-(3- dimethylaminopropyl) carbodiimide (EDC), N-hydroxysuccinimide (NHS), BS3 crosslinker, phosphate buffered saline (PBS), HEPES, Glycine, and Streptavidin DyLight™ 550 Conjugated, were purchased from Thermo Fischer Scientific, Japan. Biotin-SP-conjugated AffiniPure goat anti-mouse antibody was purchased from Jackson ImmunoResearch labs, USA. Biotinylated aptamers specific to Interleukin6 (IL6) were obtained from BasePair Biotechnologies, USA. Recombinant human IL6 (PHC0066) was purchased from Life Technologies. Mouse anti-C Reactive Protein antibody [C5] ab8279 (Abcam, Japan) and recombinant human C-reactive protein (hCRP) were obtained from Oriental Yeast Co., Ltd., Japan. Alexa Fluor 488 conjugated chicken anti-goat, Alexa Fluor 546 conjugated rabbit anti-mouse and goat anti-chicken Immunoglobulins (IgGs) were purchased from Abcam, Japan.

### INDUSTRIAL APPLICABILITY

The present invention of a simple aqueous based microcontact printing (μCP) method can create stable micro- and nanopatterns of (3-aminopropyl)triethoxysilane (APTES) on glass substrates of microfluidic devices with feature sizes ranging from a few hundred microns to 200 nm (for the first time). By combining our surface patterning technique with sensing technologies, highly sensitive bioassay systems at nanoscale can be developed in the near future.

1. An array of biomolecules comprising a substrate and a probe molecule, wherein the surface of the substrate has patterned nano features of silane.

2. The array of biomolecules according to claim 1, wherein the unpatterned surface of the substrate is blocked with PEG-silane.

3. The array of biomolecules according to claim 1, wherein the diameter of the nano features is patterned from 10 nm to 1000 nm.

4. The array of biomolecules according to claim 1, wherein the probe molecule is selected from the group consisting of proteins, peptides, antibodies, nucleic acids, carbohydrates and lipids.

5. The array of biomolecules according to claim 1, wherein the probe molecule is conjugated onto nano features of silane on the substrate.

6. A kit comprising a substrate and a probe molecule, wherein the surface of the substrate has patterned nano features of silane.

7. The kit according to claim 6, wherein the unpatterned surface of the substrate is blocked with PEG-silane.

8. The kit according to claim 6, wherein the diameter of the nano features is patterned from 10 nm to 1000 nm.

9. The kit according to claim 6, wherein the probe molecule is selected from the group consisting of proteins, peptides, antibodies, nucleic acids, carbohydrates and lipids.

10. A substrate for an array of biomolecules, wherein the surface of the substrate has patterned nano features of silane for conjugating a probe molecule.

11. The substrate for an array of biomolecules according to claim 10, wherein the unpatterned surface of the substrate is blocked with PEG-silane.

12. The substrate for an array of biomolecules according to claim 10, wherein the diameter of the nano features is patterned from 10 nm to 1000 nm.

13. The substrate for an array of biomolecules according to claim 10, wherein the biomolecule is selected from the group consisting of proteins, peptides, antibodies, nucleic acids, carbohydrates and lipids.

14. A method of micro or nano patterning of biomolecules in a multiplex format comprising the steps of
covering a silicone wafer (Si Wafer) which has nanoholes with PDMS;
separating the PDMS from the Si Wafer;
covering the PDMS with a photo-sensitive polymer;
exposing the photo-sensitive polymer to light;
separating the photo-sensitive polymer replica from the PDMS;
contacting the photo-sensitive polymer replica which was plasma activated with a planar PDMS stamp which was incubated with silane;
separating the flat PDMS to lift-off silane in the contact areas;
printing the flat PDMS with nanoholes of silane onto a glass slide;
separating the flat PDMS from the glass slide;
incubating the patterned silane with an aqueous PEG silane to block the unpatterned surface; and then
incubating the patterned/blocked APTES silane with desired biomolecule.

15. The method of claim 14 wherein the biomolecule is selected from the group consisting of proteins, peptides, antibodies, nucleic acids, carbohydrates and lipids.

16. The method of claim 14 wherein the diameter of nanoholes is patterned from 10 nm to 1000 nm.
